(19) United States
(12) Patent Application Publication
(10) Pub. No.: US 2024/0399034 A1
(43) Pub. Date: Dec. 5, 2024

(54) VACUUM DEFLATION STRUCTURE, VACUUM PUMP ASSEMBLY AND BREAST PUMP

(71) Applicant: Shenzhen TPH Technology Co., Ltd., Shenzhen (CN)

(72) Inventor: Xianzi CHEN, Shenzhen (CN)

(21) Appl. No.: 18/240,171

(22) Filed: Aug. 30, 2023

(30) Foreign Application Priority Data
Jun. 5, 2023 (CN) 202310657132.0

(51) Int. Cl. A61M 1/06; A61M 1/00; F04B 43/067

(52) U.S. Cl. CPC A61M 1/062; A61M 1/67; F04B 43/067

(57) ABSTRACT

The present disclosure discloses a vacuum deflation structure, a vacuum pump assembly and a breast pump. The vacuum deflation structure includes an operating cavity having a deflating opening, a scaling baffle occluding said deflating opening, and a deflation transmission assembly pushing said scaling baffle to move away from said deflating opening, wherein said deflation transmission assembly is forced by the pressure difference between the inside and outside of said operating cavity or by the pump shaft of a vacuum pump. The breast pump has a milk collecting casing, a diaphragm and a vacuum pump assembly. The vacuum pump is used to vacuum the scaled chamber formed by the diaphragm to deform it, the deflating opening leads to the sealed chamber where the diaphragm is located, and the sealed chamber gradually returns to its original shape when the deflating opening is opened.

### CROSS-REFERENCE TO RELATED PATENT APPLICATION

This application claims priority to and the benefit of Chinese Patent Application No. CN202310657132.0 filed in China on Jun. 5, 2023. The disclosure of the above application is incorporated herein in its entirety by reference.

### TECHNICAL FIELD

The present disclosure relates to the technical field of vacuum equipment, in particular to a vacuum deflation structure, a vacuum pump assembly and a breast pump.

### BACKGROUND

The background description provided herein is for the purpose of generally presenting the context of the disclosure. Work of the presently named inventors, to the extent it is described in this background section, as well as aspects of the description that may not otherwise qualify as prior art at the time of filing, are neither expressly nor impliedly admitted as prior art against the present disclosure.

The breast pump is a familiar electric device that makes use of a vacuum pump to function as itself. At present, the vacuum deflation function for breast pumps on the market is effectuated by means of solenoid valves, which is connected with the vacuum pump through the external pipeline. When the solenoid valve is closed, the vacuum pump draws the air in the sealed chamber for vacuuming, thus the external air cannot enter the sealed chamber through the solenoid valve; when the solenoid valve is opened, the vacuum pump stops vacuuming, and the external air enters the sealed chamber through the solenoid valve.

The internal structure of the existing solenoid valve is mainly composed of a coil, a metallic piston rod and a spring, setting up an electric circuit in the coil will cause a magnetic force on the metallic piston rod, and the alternating force arising from magnetism and elasticity on the metallic piston rod will drive the metallic piston rod to perform a repeated piston movement, functioning as deflation after opening and sealing after closing. When the coil set inside this solenoid valve is energized, it will generate a lot of heat, which will affect the feeling of using the breast pump (especially for a wearable breast pump), moreover, the coil being energized will consume more battery power, reducing the duration of the breast pump.

### SUMMARY

In order to solve the defects in the prior art such as heat generation and large power consumption caused by the use of solenoid valve for deflation, the present disclosure provides a vacuum deflation structure, a vacuum pump assembly and a breast pump, so as to achieve deflation and come into the effect such as decrease in heat and costs, by using the pressure difference between the inside and outside of the operating cavity or the pump shaft of the vacuum pump to provide motive force.

The technical scheme adopted in the present disclosure is as follows: to design a vacuum deflation structure, comprising an operating cavity having a deflating opening, a sealing baffle occluding the deflating opening, and a deflation transmission assembly pushing the sealing baffle to move away from the deflating opening, wherein the deflation transmission assembly is forced by the pressure difference between the inside and outside of the operating cavity or by the pump shaft of a vacuum pump.

In some embodiments, the operating cavity is composed of a piston chamber and a deflation cavity, one end of the piston chamber is an air inlet end communicating with the external environment, the other end of the piston chamber is a connection-adapted end for vacuuming, the deflation transmission assembly is movably arranged inside the piston chamber, and the area between the deflation transmission assembly and the connection-adapted end forms a sealed space, which is provided with an intermediate interface, by which the piston chamber and the deflation cavity intercommunicate with each other; the sealing baffle has a fixed portion rotationally installed and movable ends disposed on both sides of the fixed portion, the first movable end of the sealing baffle occludes the deflating opening, the second movable end of the sealing baffle extends into the sealed space from the intermediate interface, the deflation transmission assembly can move to contact with the second movable end and poke the sealing baffle to rotate, so that the first movable end moves away from the deflating opening or close to the deflating opening.

Further, the deflation transmission assembly is provided with a skewed groove for accommodating the second movable end, the skewed groove gradually deepens in the direction close to the connection-adapted end, and the deepest position of the skewed groove is provided with a deflation-stopping surface; when the deflation transmission assembly moves toward the connection-adapted end, the second movable end is jacked up by the skewed groove, so that the first movable end moves away from the deflating opening; when the deflation transmission assembly moves toward the air inlet end to be in contact with the second movable end at the deflation-stopping surface, the second movable end is tightened by the deflation-stopping surface, so that the first movable end occludes the deflating opening.

Further, the deflation transmission assembly includes a movable plug positioned in the piston chamber, a sealing ring disposed between the the piston chamber and the movable plug, and a first elastic reset member forcing the movable plug to move close to the air inlet end, the skewed groove is disposed on the outer wall of the movable plug, so that the movable plug is pushed away from the air inlet end under the inward and outward pressure difference when the pressure in the sealed space increases.

Further, the cavity wall of the piston chamber is provided with a positioning protrusion for limiting the extreme position of the movable plug, the movable plug moves toward the air inlet end, so as to reach the extreme position until in contact with the positioning protrusion, and the first movable end occludes the deflating opening when the movable plug has reached the extreme position or before the movable plug reaches the extreme position.

Further, a second elastic reset member that forces the first movable end to occlude the deflating opening is installed inside the deflation cavity.

Further, the pump shaft of the vacuum pump rotates during vacuuming, and the pump shaft of the vacuum pump stops rotating when the actual pressure of the operating cavity decreases to a set pressure.

Further, the vacuum deflation structure serving as an end cover is installed at the end of the vacuum pump or independently installed on the outside of the vacuum pump.

In some embodiments, the operating cavity is provided with a connection-adapted port for vacuuming, the deflation transmission assembly is connected to the pump shaft of the vacuum pump, a third elastic reset member that forces the sealing baffle to occlude the deflating opening is installed inside the operating cavity, the deflation transmission assembly is provided with a deflation baffle rotating with the pump shaft, and the deflation baffle can rotate to contact with the sealing baffle and push the sealing baffle to move away from the deflating opening.

Further, the deflation transmission assembly includes an eccentric shaft seat mounted on the pump shaft, and a deflation baffle, one end of which is hinged on the eccentric shaft seat, the other end of the deflation baffle extends toward the sealing baffle to form a poking rod, one side of the poking rod is provided with a wedged portion, and one side of the sealing baffle is provided with a recessed portion cooperating with the shape of the wedged portion; when the deflation baffle rotates forward with the pump shaft until the wedged portion is inserted into the recess portion, the sealing baffle is jacked up by the wedged portion, so that the sealing baffle leaves the deflating opening; when the deflation baffle rotates backward with the pump shaft until the deflation baffle is in contact with the sealing baffle, the poking rod is occluded by the sealing baffle, so that the deflation baffle rotates to dodge the sealing baffle.

Further, the operating cavity is provided with limit protrusions located on both sides of the sealing baffle, so that the sealing baffle can only move in a straight line between the two limit protrusions to move away from the deflating opening or close to the deflating opening.

Further, the pump shaft of the vacuum pump rotates backward during vacuuming, and the pump shaft of the vacuum pump rotates forward when the actual pressure of the operating cavity decreases to a set pressure.

Further, the vacuum deflation structure is embedded inside the vacuum pump.

The present disclosure further provides a vacuum pump assembly, comprising a vacuum pump and the vacuum deflation structure according to the abovesaid.

The present disclosure further provides a breast pump, comprising the vacuum pump assembly.

Further, the breast pump further includes a milk collecting casing and a diaphragm, the vacuum pump is used to vacuum the sealed chamber formed by the diaphragm to deform it, the deflating opening leads to the sealed chamber where the diaphragm is located via the operating cavity, and the sealed chamber gradually returns to its original shape when the deflating opening is opened.

Compared with the prior art, the present disclosure has the following beneficial effects:
1. The present disclosure can effectively decrease the heat generation of the breast pump during operation and improve the user's feeling, by using the pressure difference between the inside and outside of the operating cavity or the pump shaft of the vacuum pump to provide motive force.
2. The present disclosure adopts a design of a transmission assembly for deflation based on mechanical structure transmission, replacing the solenoid valve in the prior art with structural components, decreasing the number of electronic control members of the breast pump, and effectively reducing production costs.
3. The present disclosure makes use of the pressure difference between the inside and outside of the operating cavity to achieve deflation without the need for additional electrical energy, enabling tangible decrease in the battery power consumption and sharp increase in the duration of the breast pump.
4. The vacuum deflation structure can be embedded and installed inside the vacuum pump, without additional external connection pipes, making full use of the design space, decreasing the overall volume of the breast pump, and easing carrying and using.

### BRIEF DESCRIPTION OF THE DRAWINGS

We shall describe the present disclosure in detail as follows in combination with examples and drawings, where:

FIG. 1 is a front view of the breast pump according to the present disclosure.

FIG. 2 is a partial exploded view of the breast pump according to the present disclosure.

FIG. 3 is an assembly diagram of the vacuum pump assembly in Example 1.

FIG. 4 is an assembly diagram of the vacuum deflation structure in Example 1.

FIG. 5 is a section diagram of the vacuum deflation structure in Example 1.

FIG. 6 is a section diagram of the deflation transmission assembly in Example 1.

FIG. 7 is an operational flowchart of the breast pump in Example 1.

FIG. 8 is an assembly diagram of the vacuum pump assembly in Example 2.

FIG. 9 is a section diagram of the vacuum pump assembly in Example 2.

FIG. 10 is a partial exploded view of the vacuum deflation structure in Example 2.

FIG. 11 is a partial exploded view of the vacuum deflation structure in Example 2.

FIG. 12 is an operational flowchart of the breast pump in Example 2.

where 100—vacuum deflation structure; 1—operating cavity; 11—deflating opening; 12—piston chamber; 13—deflation cavity; 14—air inlet hole; 15—irst air-bleed port; 16—second air-bleed port; 17—econd elastic reset member; 18—connection-adapted port; 19—exhaust port; 101—spring support; 102—limit protrusion; 2—sealing baffle; 21—first movable end; 22—second movable end; 23—recessed portion; 3—deflation transmission assembly; 31—movable plug; 311—skewed groove; 3111—deflation-stopping surface; 32—sealing ring; 33—first elastic reset member; 34—deflation baffle; 341—poking rod; 3411—wedged portion; 35—eccentric shaft seat; 4—third elastic reset member; 200—vacuum pump; 300—breast pump; 301—milk collecting casing; 302—diaphragm; 303—horn cover; 304—check valve.

### DETAILED DESCRIPTION OF SOME EMBODIMENTS

In order to make the technical problem to be solved by the present disclosure, the technical solution and the beneficial effects clearer, we shall further describe the present disclosure in detail in combination with the drawings and examples. It should be understood that the specific examples described herein are only intended to explain the present disclosure and are not intended to pose a limitation on the present disclosure.

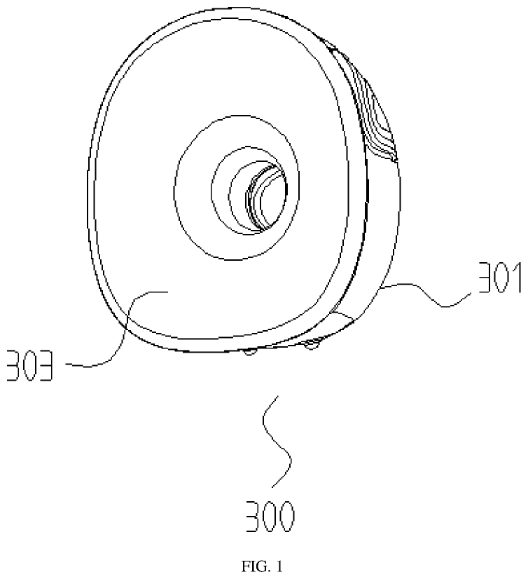

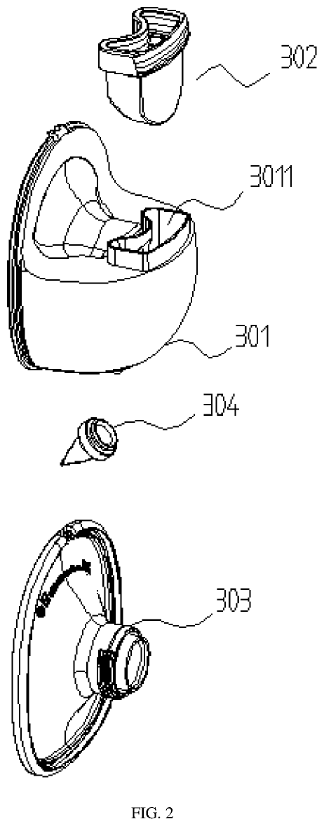

As shown in FIGS. 1-3, the vacuum deflation structure provided by the present disclosure is applied in the electric device that makes use of a vacuum pump to function as itself. The electric device includes, but is not limited to, a breast pump. The breast pump 300 typically has the horn cover 303, the milk collecting casing 301, the diaphragm 302, the vacuum pump 200 and other parts. The vacuum deflation structure 100 and the vacuum pump 200 cooperate with each other to form a vacuum pump assembly, which is typically installed on the upper back of the milk collecting casing 301. After the vacuum pump 200 has created vacuum in the sealed chamber where the diaphragm 302 is located, the vacuum deflation structure 100 performs deflation, so that the sealed chamber returns to its original shape.

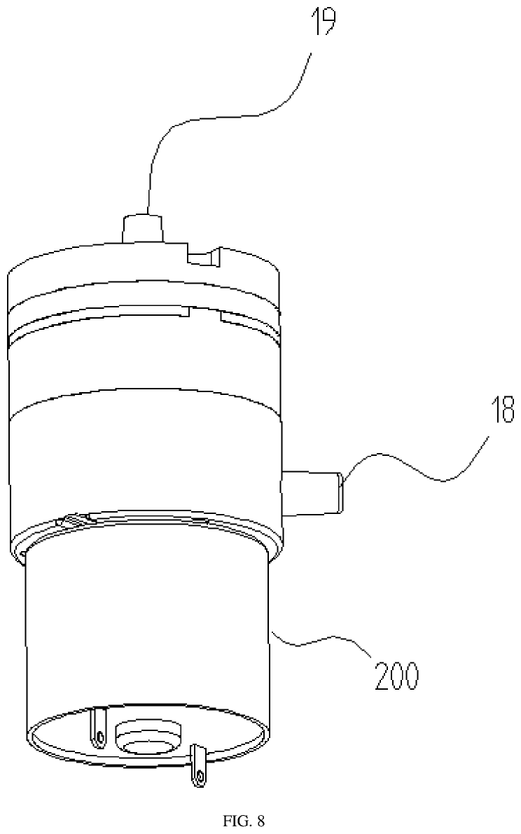

As shown in FIGS. 4-5, 8-9, the vacuum deflation structure 100 mainly includes the operating cavity 1, the sealing baffle 2 and the deflation transmission assembly 3. The operating cavity 1 has the deflating opening 11, which is occluded by the sealing baffle 2. The deflation transmission assembly 3 pushes the sealing baffle 2 away from the deflating opening 11 for deflation, and the deflation transmission assembly 3 is forced by the pressure difference between the inside and outside of the operating cavity 1 or by the pump shaft of the vacuum pump 200.

Now, we shall just describe two possible examples of the present disclosure in detail as follows.

Example 1-using the pressure difference between the inside and outside of the operating cavity to provide motive force to the deflation transmission assembly.

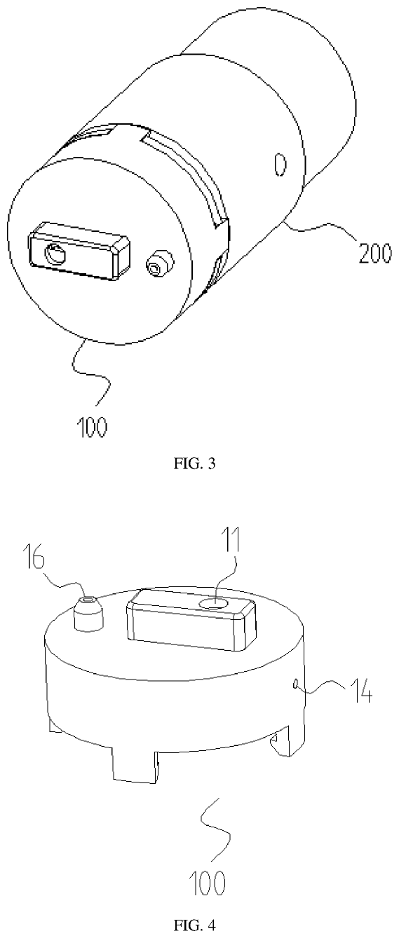

The vacuum deflation structure 100 serving as an end cover is installed at the end of the vacuum pump 200 or independently installed on the outside of the vacuum pump 200. FIG. 3 shows the vacuum deflation structure 100 serving as an end cover being installed at the end of the vacuum pump 200. The housing of the vacuum deflation structure 100 is provided with a plurality of fasteners trapping the end of the vacuum pump 200. The vacuum deflation structure 100 may also be independently installed outside the vacuum pump 200. The vacuum deflation structure 100 leads to the vacuum pump 200 and the sealed chamber via an external line. The design principles and structures for such two installation methods are basically the same with each other, we shall and describe them in detail as follows.

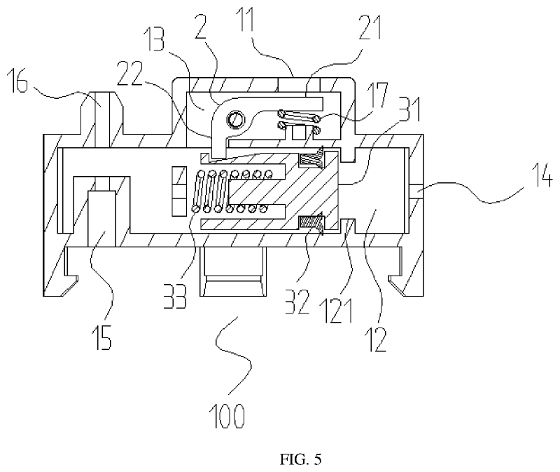

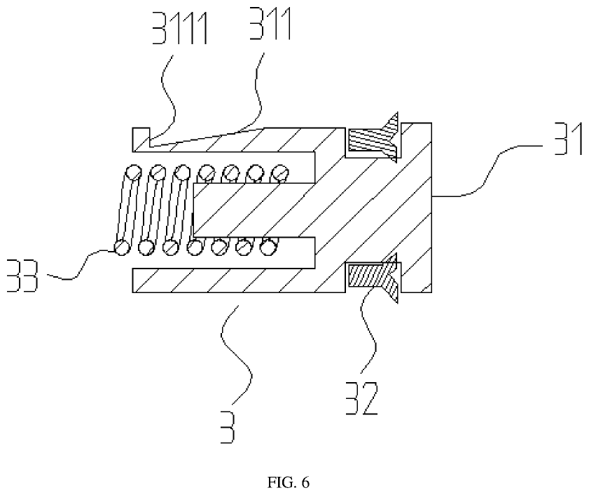

As shown in FIGS. 4 and 5, the operating cavity 1 is composed of a piston chamber 12 and a deflation cavity 13, one end of the piston chamber 12 is an air inlet end (the right end of the piston chamber is shown in FIG. 5), the air inlet end is provided with an air inlet hole 14 communicating with the external environment, which may be designed as a small hole to prevent foreign matter from entering the channel. The other end of the piston chamber 12 is a connection-adapted end (the left end of the piston chamber is shown in FIG. 5), the connection-adapted end is provided with two air-bleed ports for vacuuming, the first air-bleed port 15 is connected to the vacuum pump, and the second air-bleed port 16 is connected to the sealed chamber. The deflation transmission assembly 3 is movably arranged inside the piston chamber 12, and the area between the deflation transmission assembly 3 and the connection-adapted end inside the piston chamber 12 forms a sealed space, which is provided with an intermediate interface, by which the piston chamber and the deflation cavity intercommunicate with each other.

The sealing baffle 2 has a fixed portion and movable ends disposed on both sides of the fixed portion, in some embodiments, the sealing baffle 2 is L-shaped, the corner portion of the sealing baffle 2 acts as the fixed portion, and the straight side portion of the sealing baffle 2 acts as the movable end. The fixed portion is installed inside the deflation cavity 13 by means of a rotating shaft, and the sealing baffle 2 can be rotated by poking either of the two movable ends. The first movable end 21 of the sealing baffle 2 occludes the deflating opening 11, and the second movable end 22 of the sealing baffle 2 extends into the sealed space from the intermediate interface. The deflation transmission assembly 3 can move to contact with the second movable end 22 and poke the sealing baffle 2 to rotate, so that the first movable end 21 moves away from the deflating opening 11 or close to the deflating opening 11.

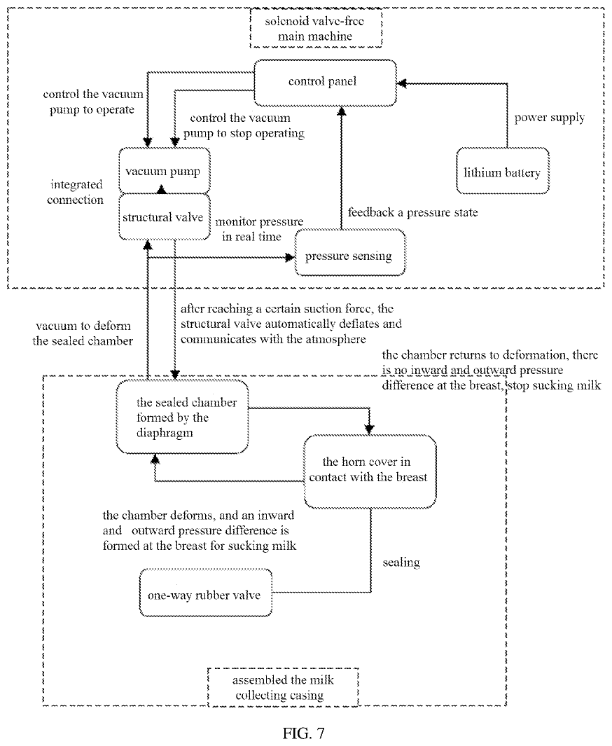

As shown in FIGS. 5 and 6, the deflation transmission assembly 3 is provided with the skewed groove 311 for accommodating the second movable end 22, the skewed groove 311 gradually deepens in the direction close to the connection-adapted end, the deepest position of the skewed groove 311 is provided with the deflation-stopping surface 3111, the slope of the skewed groove 311 influences the angle of the deflating opening 11 opened by the sealing baffle 2. When the deflation transmission assembly 3 moves toward the connection-adapted end, the depth of the skewed groove 311 in contact with the second movable end 22 gradually lessens, the second movable end 22 is jacked up, and the sealing baffle 2 rotates, so that the first movable end 21 moves away from the deflating opening 11. The closer the deflation transmission assembly 3 is to the connection-adapted end, the bigger the angle of rotation of the sealing baffle 2, and the larger the deflating opening 11, so that the amount of deflation will increase. When the deflation transmission assembly 3 moves toward the inlet end to be in contact with the second movable end 22 at the deflation-stopping surface 3111, the second movable end 22 is tightened by the deflation-stopping surface 3111, and the sealing baffle 2 rotates, so that the first movable end 21 occludes the deflating opening 11. In some embodiments, the second elastic reset member 17 that forces the first movable end 21 to occlude the deflating opening 11 is installed inside the deflation cavity 13, and the second elastic reset member 17 acts as an auxiliary elastic sealing role, so this can be done just by selecting a spring providing small force.

Specifically, as shown in FIG. 6, the deflation transmission assembly 3 includes the movable plug 31, the sealing ring 32, and the first elastic reset member 33. The movable plug 31 reciprocates in the piston chamber 12, the sealing ring 32 encircles the movable plug 31 to isolate the sealed space from the air inlet end, the first elastic reset member 33 is installed inside the piston chamber 12, providing a restoring force to the movable plug 31, so as to force the movable plug 31 to move close to the air inlet end, and the skewed groove 311 is disposed on the outer wall of the movable plug 31, so that the movable plug 31 is pushed away from the air inlet end under the inward and outward pressure difference when the pressure in the sealed space increases, and the movable plug 31 is pushed close to the air inlet end by the first elastic reset member 33 when the pressure in the sealed space decreases. In this way, the skewed groove 311 moves with the movable plug 31, thereby achieving deflating and sealing.

As shown in FIG. 5, in order to enhance the reliability of the structure, the cavity wall of the piston chamber is provided with the positioning protrusion 121 for limiting the extreme position of the movable plug 31, the movable plug 31 moves toward the air inlet end, so as to reach the extreme position until in contact with the positioning protrusion 121. The first movable end 21 occludes the deflating opening 11 when the movable plug 31 has reached the extreme position or before the movable plug 31 reaches the extreme position, ensuring that the sealing baffle 2 can be tightened and fit on the deflating opening 11.

Taking a spring used as the first elastic reset member 33 as an example, its spring force is calculated as follows:

According to ISO standard Pascal (1 pa=1 N/m²), an inquiry gives a result of 1 standard atmospheric pressure=760 mmHg=101325 pa. The negative pressure at the deflating opening in the figure may be equivalent to the negative pressure at the movable plug. Assuming that the maximum negative pressure, Max is 470 mmHg=62661.5 pa=62661.5 N/m²=62661.5 N/106 mm²=0.0626615 N/mm², and the stressed area of the movable plug, πr² is 3.14×5×5 mm²=78.5 mm², the pressure on the movable plug 31 is 0.0626615*78.5=4.919N=501.6 gf, that is, 501.6 gram-force. Then, the spring force of the first elastic reset member 33 is less than 501.6 gram-force, so that only if the vacuum pump 200 performs vacuuming and generates negative pressure, the atmospheric pressure can push the movable plug 31 to reach the maximum negative pressure.

It should be pointed out that the movement distance of the movable plug 31, the first elastic reset member 33 and the skewed groove 311 of the movable plug 31 in contact with the sealing baffle 2 can be designed according to the actual use requirements, and their stereoscopic shapes can also be changed according to the design requirements, not limited to the stereoscopic shapes of the above embodiments.

As shown in FIG. 7, in an application instance of Example 1, the operation process of the breast pump is as follows:

The milk collecting casing 301 of the breast pump 300 is in contact with the breast in an airtight state, the control panel controls the vacuum pump 200 to operate, the vacuum pump 200 vacuums the sealed chamber formed by the diaphragm 302, the diaphragm 302 deforms so as to cause decrease in the pressure inside the milk collecting casing 301 and form an inward and outward pressure difference at the breast for sucking milk, after the pressure of the sealed chamber decreases to a certain pressure, the movable plug 31 moves under the action of the inward and outward pressure difference, and the sealing baffle 2 rotates to open the deflating opening 11. The pressure sensor monitors the pressure state of the sealed chamber in real time and feeds back to the control panel, which controls the vacuum pump 200 to stop operating when the pressure decreases to a set pressure, the outside air enters the sealed chamber from the deflating opening 11, so that the pressure of the sealed chamber returns to its normal state, the diaphragm 302 returns to its original shape, so that the pressure inside the milk collecting casing 301 increases, then, there is no inward and outward pressure difference at the breast, stopping sucking milk, the movable plug 31 gradually resets as the pressure in the sealed chamber returns to its normal state, and the sealing baffle 2 turns to seal the deflating opening 11.

The vacuum deflation structure 100 makes use of the pressure difference inside and outside the operating cavity 1 to repeatedly perform the cycle operation such as sealing (vacuuming)→deflating→sealing (vacuuming), the whole cycle process simulates baby's sucking motion, spurring the milk to flow out of the breast.

Example 2—using the pump shaft of the vacuum pump to provide motive force to the deflation transmission assembly.

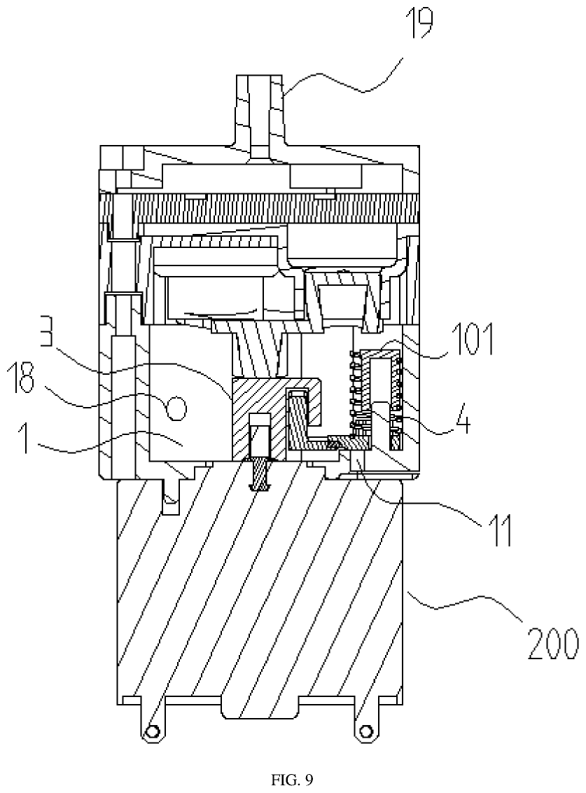

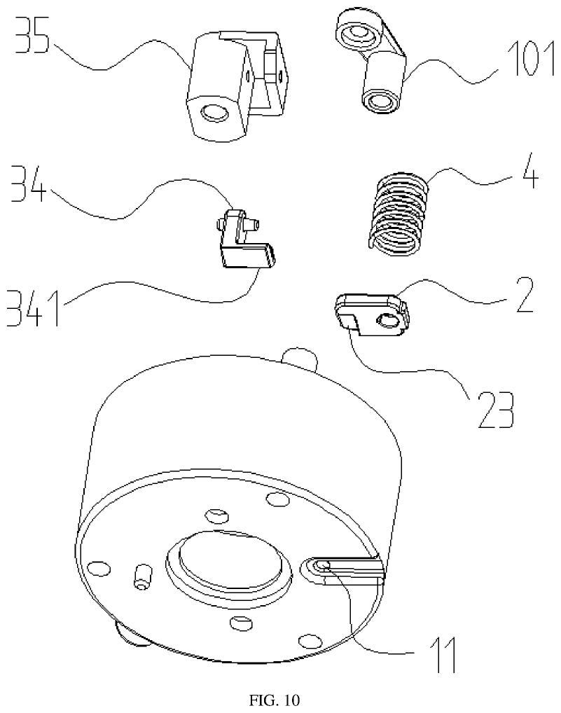

The vacuum deflation structure 100 is embedded inside the vacuum pump 200, FIG. 9 shows the internal structure inside the vacuum pump 200 of the vacuum deflation structure 100, we shall describe it in detail as follows.

As shown in FIGS. 9 and 10, the operating cavity 1 is provided with the connection-adapted port 18 for vacuuming, which is connected to the sealed chamber, the deflation transmission assembly 3 is connected to the pump shaft of the vacuum pump 200, the third elastic reset member 4 that forces the sealing baffle to occlude the deflating opening is installed inside the operating cavity 1, the third elastic return member 4 is fixedly positioned by the spring support 101, and the deflation transmission assembly 3 is provided with the deflation baffle 34 rotating with the pump shaft, the deflation baffle 34 can rotate to contact with the sealing baffle 2 and push the sealing baffle 2 to move away from the deflating opening 11.

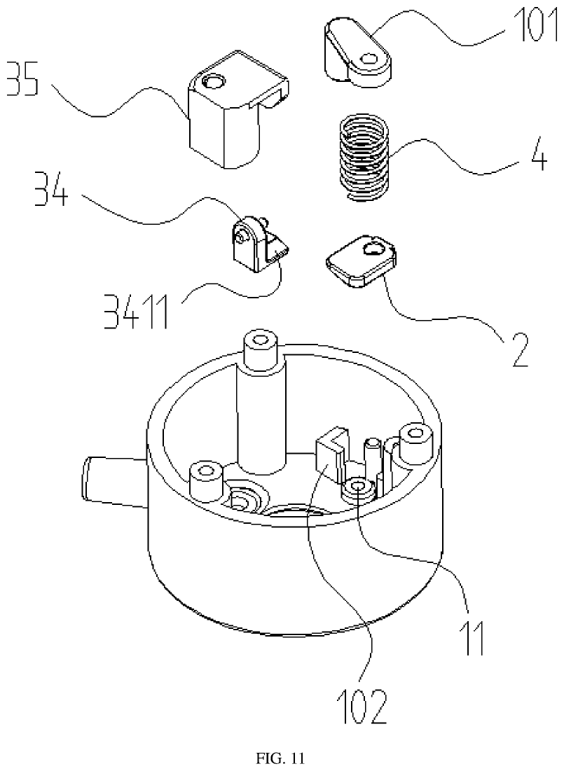

As shown in FIGS. 10 and 11, the deflation transmission assembly 3 includes the eccentric shaft seat 35 and the deflation baffle 34, the eccentric shaft seat 35 is mounted on the pump shaft, one end of the deflation baffle 34 is hinged on the eccentric shaft seat 35, and the other end of the deflation baffle 34 extends toward the sealing baffle 2 to form a poking rod. In some embodiments, the deflation baffle 34 is L-shaped, the straight edge of the deflation baffle 34 is hinged on the eccentric shaft seat 35, and its other straight edge is the poking rod 341. One side of the poking rod 341 is provided with the wedged portion 3411, and one side of the sealing baffle 2 is provided with the recessed portion 23 cooperating with the shape of the wedged portion 3411. When the deflation baffle 34 rotates forward with the pump shaft until the wedged portion 3411 is inserted into the recess portion 23, the sealing baffle 2 is jacked up by the wedged portion 3411, so that the sealing baffle 2 leaves the deflating opening 11, thus the deflating opening 11 is opened; the wedged portion 3411 jacks up the sealing baffle 2, and then continues to rotate with the pump shaft until it leaves the sealing baffle 2, the sealing baffle 2 returns to its original position under the action of the third elastic reset member 4, thus the deflating opening 11 is closed. Each time the deflation baffle 34 rotates one round with the pump shaft, the deflating opening 11 is opened once, and the deflation time is the duration that the sealing baffle 2 keeps being jacked up. When the deflation baffle 34 rotates backward with the pump shaft until the deflation baffle 34 is in contact with the sealing baffle 2, the poking rod 341 is occluded by the sealing baffle 2, so that the deflation baffle 34 rotates along the articulated shaft on the eccentric shaft seat 35 to dodge the sealing baffle 2, the deflation baffle 34 dodges the sealing baffle 2 and continues to rotate with the pump shaft until it leaves it. Each time the deflation baffle 34 rotates one round with the pump shaft, the deflation baffle 34 rotates along the articulated shaft to dodge the sealing baffle 2 once, and the deflating opening 11 is always closed.

In order to prevent the sealing baffle 2 from swinging after contacting with the poking rod 341, the operating cavity 1 is provided with the limit protrusions 102 located on both sides of the sealing baffle 2, so that the sealing baffle 2 can only move in a straight line between the two limit protrusions 102 to move away from the deflating opening 11 or close to the deflating opening 11. When the poking rod 341 rotates backward with the pump shaft, the sealing baffle 2 is occluded by the limit protrusion on one side, and can only be jacked away from the deflating opening 11 by the wedged portion 3411. When the poking rod 341 rotates backward with the pump shaft, the sealing baffle 2 is occluded by the limit protrusion 102 on the other side, forcing the deflation baffle 34 where the poking rod 341 is located to turn and dodge, and ensuring the reliability of deflating and sealing.

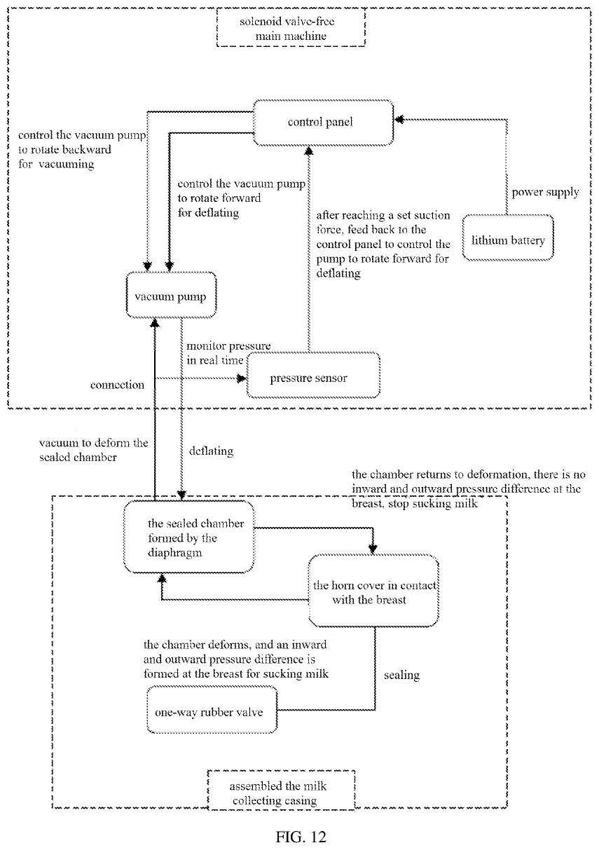

As shown in FIG. 12, in an application instance of Example 2, the operation process of the breast pump is as follows:

The milk collecting casing 301 of the breast pump 300 is in contact with the breast in an airtight state, the control panel controls the vacuum pump 200 to rotate backwards, the vacuum pump 200 vacuums the sealed chamber formed by the diaphragm 302, the diaphragm 302 deforms so as to cause decrease in the pressure inside the milk collecting casing 301 and form an inward and outward pressure difference at the breast for sucking milk. The pressure sensor monitors the pressure state of the sealed chamber in real time and feeds back to the control panel, which controls the vacuum pump 200 to rotate forwards when the pressure decreases to a set pressure, the outside air enters the sealed chamber from the deflating opening 11 when the deflating opening is opened intermittently, so that the pressure of the sealed chamber returns to its normal state, the diaphragm 302 returns to its original shape, so that the pressure inside the milk collecting casing 301 increases, then, there is no inward and outward pressure difference at the breast, stopping sucking milk.

The vacuum deflation structure 100 makes use of the vacuum pump 200 to repeatedly perform rotating backwards (vacuuming)→rotating forwards (deflating)→rotating backwards (vacuuming), the whole cycle process simulates baby's sucking motion, spurring the milk to flow out of the breast.

It should be pointed out that the elastic reset members mentioned above may be a spring, an elastic rubber member or an elastic alloy member. Since the sealing baffle 2 needs to fit on the deflating opening 11 for sealing, in order to achieve a better sealing effect, the part of the sealing baffle 2 in contact with the deflating opening 11 is coated or a sealing gasket is added between the two. Certainly, other sealing methods may also be used, and the present disclosure does not impose any special limitations on this.

As shown in FIGS. 1-3, the present disclosure also provides a vacuum pump assembly, including a vacuum pump 200 and the aforementioned vacuum deflation structure 100. The breast pump 300 described above includes the horn cover 303, the milk collecting casing 301, the diaphragm 302 and a main machine, the main machine includes a control panel, a rechargeable battery and this vacuum pump assembly, and the control panel is connected to a pressure sensor. Specifically, the horn cover 303 is embedded on the milk collecting casing 301, the milk collecting casing 301 is provided with the groove 3011 containing the diaphragm 302, the outlet end of the horn cover 303 is connected to the groove 3011. The vacuum pump 200 is used to vacuum the sealed chamber formed by the diaphragm 302 to deform it, the deflating opening 11 leads to the sealed chamber where the diaphragm 302 is located, and the sealed chamber gradually returns to its original shape when the deflating opening 11 is opened. A liquid outlet is opened on the bottom of the groove 3011, the liquid outlet is provided with the check valve 304 that only allows milk to flow out, the operating process of the breast pump 300 has been described in detail above, no need to repeat herein again. It should be understood that the main machine may be worn inside a bra or outside a bra, and the present disclosure does not impose any special limitations on the application form of the main machine.

It should be noted that the terms used above are only intended to describe specific embodiments, and are not intended to limit exemplary embodiments according to the present disclosure. When the terms “comprising” and/or “including” are used in this description, they indicate the presence of features, steps, operations, devices, assemblies, and/or combinations thereof. The sequence to be executed for actions, steps and the likes in the device and method shown in the description and drawings can be realized in any sequence, as long as there is no special explicit limitation on the sequence, as well as the output of the preceding process is not used in the subsequent process. Using the similar terms indicating sequence for convenience does not imply that they have to be implemented in such sequence.

The technology, methods and equipment known to a person skilled in the relevant art may not be discussed in detail, but where appropriate, the technology, methods and equipment shall be considered as a part of the description to be authorized. Of all the examples shown and discussed herein, any specific values should be interpreted as an exemplary value only and not as a limitation. Thus, other examples of exemplary embodiments may have different values. It should be noted that similar marks and characters indicate similar terms in the subsequent drawings, so that once an item is defined in one drawing among them, it does not need to be further discussed in subsequent drawings.

The aforementioned examples are only a better embodiment of the present disclosure and are not intended to limit the present disclosure, and any modification, equivalent replacement and improvement made within the essence and principle of the present disclosure shall be included in the protection scope of the present disclosure.

What is claimed is:

1. A vacuum deflation structure, comprising an operating cavity having a deflating opening, a sealing baffle occluding said deflating opening, and a deflation transmission assembly pushing said sealing baffle to move away from said deflating opening, wherein said deflation transmission assembly is forced by the pressure difference between the inside and outside of said operating cavity or by the pump shaft of a vacuum pump.

2. The vacuum deflation structure according to claim 1, wherein said operating cavity is composed of a piston chamber and a deflation cavity, one end of said piston chamber is an air inlet end communicating with the external environment, the other end of said piston chamber is a connection-adapted end for vacuuming, said deflation transmission assembly is movably arranged inside said piston chamber, and the area between said deflation transmission assembly and said connection-adapted end forms a sealed space, which is provided with an intermediate interface, by which said piston chamber and said deflation cavity intercommunicate with each other;
wherein said sealing baffle has a fixed portion rotationally installed and movable ends disposed on both sides of said fixed portion, the first movable end of said sealing baffle occludes said deflating opening, the second movable end of the sealing baffle extends into said sealed space from said intermediate interface, said deflation transmission assembly can move to contact with said second movable end and poke said sealing baffle to rotate, so that said first movable end moves away from said deflating opening or close to said deflating opening.

3. The vacuum deflation structure according to claim 2, wherein said deflation transmission assembly is provided with a skewed groove for accommodating said second movable end, said skewed groove gradually deepens in the direction close to said connection-adapted end, and the deepest position of said skewed groove is provided with a deflation-stopping surface;
when said deflation transmission assembly moves toward said connection-adapted end, said second movable end is jacked up by said skewed groove, so that said first movable end moves away from said deflating opening;
when said deflation transmission assembly moves toward the air inlet end to be in contact with said second movable end at said deflation-stopping surface, said second movable end is tightened by said deflation-stopping surface, so that said first movable end occludes said deflating opening.

4. The vacuum deflation structure according to claim 3, wherein said deflation transmission assembly includes a movable plug positioned in said piston chamber, a sealing ring disposed between said the piston chamber and said movable plug, and a first elastic reset member forcing said movable plug to move close to said air inlet end, said skewed groove is disposed on the outer wall of said movable plug, so that said movable plug is pushed away from said air inlet end under the inward and outward pressure difference when the pressure in the sealed space increases.

5. The vacuum deflation structure according to claim 4, wherein the cavity wall of said piston chamber is provided with a positioning protrusion for limiting the extreme position of said movable plug, said movable plug moves toward said air inlet end, so as to reach the extreme position until in contact with said positioning protrusion, and said first movable end occludes said deflating opening when said movable plug has reached the extreme position or before said movable plug reaches the extreme position.

6. The vacuum deflation structure according to claim 2, wherein a second elastic reset member that forces said first movable end to occlude said deflating opening is installed inside said deflation cavity.

7. The vacuum deflation structure according to claim 2, wherein the pump shaft of said vacuum pump rotates during vacuuming, and the pump shaft of said vacuum pump stops rotating when the actual pressure of said operating cavity decreases to a set pressure.

8. The vacuum deflation structure according to claim 2, wherein said vacuum deflation structure serving as an end cover is installed at the end of said vacuum pump or independently installed on the outside of said vacuum pump.

9. The vacuum deflation structure according to claim 1, wherein said operating cavity is provided with a connection-adapted port for vacuuming, said deflation transmission assembly is connected to the pump shaft of said vacuum pump, a third elastic reset member that forces said sealing baffle to occlude said deflating opening is installed inside said operating cavity, said deflation transmission assembly is provided with a deflation baffle rotating with the pump shaft, and said deflation baffle can rotate to contact with said sealing baffle and push said sealing baffle to move away from said deflating opening.

10. The vacuum deflation structure according to claim 9, wherein said deflation transmission assembly includes an eccentric shaft seat mounted on the pump shaft, and a deflation baffle, one end of which is hinged on said eccentric shaft seat, the other end of said deflation baffle extends toward said sealing baffle to form a poking rod, one side of said poking rod is provided with a wedged portion, and one side of said sealing baffle is provided with a recessed portion cooperating with the shape of said wedged portion;
when the deflation baffle rotates forward with the pump shaft until said wedged portion is inserted into said recess portion, said sealing baffle is jacked up by said wedged portion, so that said sealing baffle leaves said deflating opening;
when said deflation baffle rotates backward with the pump shaft until said deflation baffle is in contact with said sealing baffle, said poking rod is occluded by said sealing baffle, so that said deflation baffle rotates to dodge said sealing baffle.

11. The vacuum deflation structure according to claim 10, said operating cavity is provided with limit protrusions located on both sides of said sealing baffle, so that said sealing baffle can only move in a straight line between the two limit protrusions to move away from said deflating opening or close to said deflating opening.

12. The vacuum deflation structure according to claim 9, wherein the pump shaft of said vacuum pump rotates backward during vacuuming, and the pump shaft of said vacuum pump rotates forward when the actual pressure of said operating cavity decreases to a set pressure.

13. The vacuum deflation structure according to claim 12, wherein said vacuum deflation structure is embedded inside said vacuum pump.

14. A vacuum pump assembly, comprising a vacuum pump and the vacuum deflation structure according to claim 1.

15. A breast pump, comprising the vacuum pump assembly according to claim 14.

16. The breast pump according to claim 15, wherein the breast pump further includes a milk collecting casing and a diaphragm, said vacuum pump is used to vacuum said sealed chamber formed by said diaphragm to deform it, said deflating opening leads to said sealed chamber where said diaphragm is located via said operating cavity, and said sealed chamber gradually returns to its original shape when said deflating opening is opened.
